(19) United States
(12) Patent Application Publication
(10) Pub. No.: US 2003/0045580 A1
(43) Pub. Date: Mar. 6, 2003

(54) Ibuprofen containing active agent preparation

(76) Inventor: Heinz Einig, Neustadt (DE)

Correspondence Address: Keil & Weinkauf, 1350 Connecticut Ave NW, Washington, DC, 20036, US

(21) Appl. No.: 10/181,459

(22) PCT Filed: Jan. 19, 2001
(86) PCT No.: PCT/EP01/00602

(30) Foreign Application Priority Data
Jan. 28, 2000 (DE) 10003757.7

(51) Int. Cl. A61K 31/192; A61K 9/20

(52) U.S. Cl. 514/570; 424/465

(57) ABSTRACT

The present invention relates to an active ingredient preparation comprising ibuprofen, to a process for its production, to its use for producing pharmaceutical preparations and solid dosage forms, and to pharmaceutical preparations and solid dosage forms comprising the ibuprofen active ingredient preparation.

The present invention relates to an active ingredient preparation comprising ibuprofen, to a process for its production, to its use for producing pharmaceutical preparations and solid dosage forms, and to pharmaceutical preparations and solid dosage forms comprising the ibuprofen active ingredient preparation.

Ibuprofen, 2-(4-isobutylphenyl)propionic acid, is a known active ingredient with antiinflammatory, analgesic and antipyretic effect which is mainly employed for treating pain and for inhibiting inflammation.

Because of the poor compressibility and the low melting point of the known ibuprofen crystal modifications, racemic ibuprofen and, in particular, the S enantiomer which has better and faster activity display poor tableting properties and can be converted into sufficiently stable solid dosage forms, such as, for example, tablets, only with the assistance of large amounts of pharmaceutical excipients.

Ibuprofen is usually administered in tablets containing 200 or 400 mg of active ingredient/tablet, but for rheumatic complaints also in doses of up to 600 and 800 mg of active ingredient/tablet.

Owing to the admixture of large amounts of pharmaceutical excipients, the active ingredient concentration in solid dosage forms is limited to about 30 to 40% by weight. On the one hand, this leads to higher production costs for each quantity of active ingredient, and it has the further disadvantage that solid dosage forms with high doses of active ingredient can scarcely be swallowed owing to their size.

It is possible by the further processing step of a wet or dry granulation of the pharmaceutical preparation before tableting to reduce the quantity of pharmaceutical excipients, so that active ingredient concentrations of up to 66% are reached in the solid dosage forms.

However, this step is very time-consuming and costly. In addition, the process has the disadvantage that a formula for solid dosage forms with high doses of active ingredient cannot be easily applied to smaller solid dosage forms with lower doses of active ingredient. The smaller solid dosage forms usually have a hardness which is no longer adequate.

The state of the art nowadays for reducing costs is a superfast and flexible tableting. For this it is necessary to be able to employ pharmaceutical preparations flexibly for solid dosage forms varying in size and dose of active ingredient.

A cost-saving process for producing solid dosage forms is a so-called direct tableting. This entails all the ingredients of a tablet being mixed in a single step to give the composition which is ready for tableting and then being compressed to the finished tablet. It is possible in this way to avoid a pregranulation step.

For this purpose it is necessary to convert ibuprofen into a form which can be tableted directly.

It is known to convert ibuprofen into an ibuprofen-containing active ingredient preparation which can be tableted directly with the remaining ingredients of the tablet such as, for example, pharmaceutical aids.

Advantageous ibuprofen-containing active ingredient preparations for direct tableting display as many as possible of the following properties:

The active ingredient preparation can be comressed directly to solid dosage forms with adequate hardness.

The active ingredient preparation has a high ibuprofen concentration.

The solid dosage forms produced from the active ingredient preparation have a high ibuprofen concentration, i.e. as few further pharmaceutical aids as possible are necessary to produce advantageous solid dosage forms from the active ingredient preparation.

The solid dosage forms produced from the active ingredient preparation display adequate hardness and friability.

The solid dosage forms produced from the active ingredient preparation disintegrate and release the active ingredient ibuprofen in solution within the required time (instant release/sustained release)

The solid dosage forms produced from the active ingredient preparation are stable on storage.

The pharmaceutical preparations mixed from the active ingredient preparation can be used flexibly for different sizes of the dosage forms and permit superfast tableting.

WO 89/02266 describes a process for producing an ibuprofen-containing pharmaceutical formulation which can be tableted by spraying an aqueous mixture of ibuprofen and sodium carboxymethylcellulose with a dispersion of pregelatinized starch in a fluidized bed granulation/drying process, subsequently drying and further admixing a lubricant, it being possible to admix further pharmaceutical excipients before and after the spraying. The active ingredient preparation produced in the process reaches a maximum ibuprofen concentration of 88.5% by weight. The pharmaceutical preparations and tablets resulting therefrom accordingly have a maximum ibuprofen concentration of only 85% by weight. Tablets with optimal hardness and active ingredient release produced by this process in fact have a maximum ibuprofen concentration of only 63% by weight. The process and the pharmaceutical formulation produced thereby thus have the disadvantage that tablets with ibuprofen concentrations higher than 85% by weight cannot be produced.

EP 456 720 describes an active ingredient preparation which contains 85 to 99% by weight of ibuprofen and can be directly tableted after addition of further pharmaceutical excipients. For this purpose, ibuprofen is mixed with a binder (but not polyvinylpyrrolidone) and fluidized and granulated with a dispersion of polyvinylpyrrolidone (PVP) and further binders in a spray drying process. This process and the active ingredient preparation produced thereby have the disadvantage that uncrosslinked polyvinylpyrrolidone is used as granulating agent or as film-forming binder in the granulation process. Uncrosslinked polyvinylpyrrolidone forms with ibuprofen at temperatures from 30 to 40° C. a eutectic phase which is liquid at room temperature. This leads to heavy deposits on the tablet punches, so that superfast tableting is no longer possible. In addition, tablets produced from this active ingredient preparation granulated with uncrosslinked PVP have the disadvantage of being less storable. At 30 to 40° C., the tablets undergo further hardening so that the active ingredient release rate is reduced.

It is an object of the present invention to provide a further ibuprofen-containing active ingredient preparation which no longer has the aforementioned disadvantages and complies with as many as possible of the aforementioned advantageous properties.

We have found that this object is achieved by a process for producing an active ingredient preparation comprising ibuprofen, wherein ibuprofen or its salt is coated with a binder apart from polyvinylpyrrolidone, where the proportion of the binder is from 0.1 to 10% by weight and the proportion of ibuprofen or its salts is from 90 to 99.9% by weight, based on the dry weight of the active ingredient preparation.

Ibuprofen means both racemic ibuprofen and the isolated enantiomers R-(−)-ibuprofen and, in particular, S-(+)-ibuprofen which has faster and better activity.

Suitable salts of ibuprofen are, in particular, the alkali metal and alkaline earth metal salts, and the salts of ibuprofen with basic amino acids such as, for example, lysine. Particularly preferred salts are ibuprofen sodium, ibuprofen calcium and ibuprofen lysine.

The particle size of the ibuprofen employed is not critical. It is typically from 10 to 100 μm, in particular 20 to 80 μm. The purity of the ibuprofen employed is likewise not critical for the invention but, with a view to a high active ingredient concentration in the solid dosage form, ought to be as high as possible, in particular greater than 96% or 99%.

Binders also mean mixtures of binders. For producing the active ingredient preparation it is possible to use all known binders but not polyvinylpyrrolidone (PVP), in particular not uncrosslinked PVP.

Examples of suitable binders are celluloses or cellulose derivatives, starch or starch derivatives, gelatin, alginate suspensions or sugar solutions.

Preferred binders are water-soluble, -suspendable or -dispersible binders. Particularly preferred binders are hydroxypropylmethylcelluloses, in particular with an average molecular weight which corresponds approximately to the commercial type Pharmacoat® 603 or 606 supplied by Shinetsu Chemical Comp., Tokyo, Japan, or grades with higher viscosity, methylcellulose, sodium carboxymethylcellulose, hydroxypropylcellulose or gelatin.

The coating of the active ingredient ibuprofen with the binder can in principle be carried out in all known ways. For this purpose the binder is usually brought into vigorous contact, in liquid or molten form, or in a solution, suspension or dispersion, with the active ingredient to be coated, and the resulting granules are subsequently dried, where appropriate after a classification and homogenization. It is then possible for the dried granules to be, for example through appropriate sieves, further classified or homogenized.

The application of the solution, suspension or dispersion of the binder can take place from organic or aqueous solution. In a preferred embodiment, aqueous solutions, suspensions or dispersions of the binder are used.

The bringing into contact can take place by mixing the active ingredient with the melt or solution, suspension or dispersion of the binder, for example in a conventional wet mixer.

The wet mixing can also take place, for example, by mixing the active ingredient and the binder dry, and then fluidizing with an organic solvent or water and kneading.

In the case of wet mixing, the drying of the resulting granules takes place separately, for example after the classification or homogenization of the wet granules in a dryer, such as, for example, an IR, circulating air or fluidized bed drier.

However, the bringing into contact can also take place by spraying the melt or solution, suspension or dispersion of the binder onto or with the active ingredient in a fluid or fluidized bed.

It is moreover possible for the bringing into contact and drying to take place in one step, for example in fluidized bed granulators.

In a preferred embodiment of the process, the coating of ibuprofen or its salts with the binder takes place in a wet mixer, for example with plowshare mixer and chopper and subsequent drying or in fluidized bed granulators with bottom or top spraying, in particular in granulators of the Wurster or Glatt-Zeller type.

Wet mixers are the most commercially economic because the active ingredient and the binder are initially only mixed and then moistened with water or an organic solvent and kneaded. Production of homogeneous wet granules can be followed by drying in a conventional drier (IR, circulating air or fluidized bed drier). The homogenization is followed by classification through sieves. A large output is achieved with this method.

In the production of the ibuprofen-containing active ingredient preparation, the proportion of the binder is from 0.1 to 10% by weight and the proportion of ibuprofen or its salts is from 90 to 99.9% by weight, in each case based on the dry weight of the active ingredient preparation.

In a preferred embodiment, the proportion of the binder is from 0.2 to 6% by weight and the proportion of ibuprofen or its salts is from 94 to 99.8% by weight, in each case based on the dry weight of the active ingredient preparation.

In a particularly preferred embodiment, the proportion of the binder is from 0.3 to 3.5% by weight and the proportion of ibuprofen or its salts is from 96.5 to 99.7% by weight, in each case based on the dry weight of the active ingredient preparation.

It is possible to add to the ibuprofen to be coated or its salts, after or, preferably, before the coating with binder, up to 1.5% by weight of adsorbent, such as, for example, Pharmasorb(R) supplied by Engelhard Minerals & Chemicals Div., Menlo Park, Edison, N.J. 08817, USA, or highly disperse silica, such as, for example, Aerosil® 200.

In the case where adsorbent is added, highly disperse silica, such as, for example, Aerosil® 200, supplied by Degussa, Frankfurt/Main, Germany, is preferred.

The present invention accordingly relates to an ibuprofen-containing active ingredient preparation obtainable by the process described above.

In contrast to a purely physical mixture which contains from 90 to 99.9% by weight of ibuprofen and from 0.1 to 10% by weight of binder, in each case based on the dry weight of the mixture, the novel active ingredient preparation can be converted into compacts having a hardness of greater than 25 N.

The invention therefore also relates to a polyvinylpyrrolidone-free active ingredient preparation comprising

| | |
|---|---|
| 90.0 to 99.9% | by weight of ibuprofen or its salts, |
| 0.1 to 10.0% | by weight of binder apart from |
| | polyvinylpyrrol idone |
| | and |
| 0.1 to 1.5% | by weight of highly disperse silica, |

in each case based on the dry weight of the active ingredient preparation,

where the compacts produced from this active ingredient preparation with a compressive force of from 4.7 to 5.3 kN and having an ibuprofen active ingredient content of 200 mg and a diameter of 9 mm have a hardness of at least 25 N.

The hardness of the round compacts means the radial breaking force.

In particular, the invention relates to an active ingredient preparation consisting of

| | |
|---|---|
| 90.0 to 99.9% | by weight of ibuprofen or its salts, |
| 0.1 to 10.0% | by weight of binder apart from |
| | polyvinylpyrrolidone |
| | and |
| 0 to 1.5% | by weight of highly disperse silica, |

in each case based on the dry weight of the active ingredient preparation,

where the compacts produced from this active ingredient preparation with a compressive force of from 4.7 to 5.3 kN and having an ibuprofen active ingredient content of 200 mg and a diameter of 9 mm have a hardness of at least 25 N.

In this case, the percentages by weight of the ingredients in the active ingredient preparation add up to 100% by weight.

The novel active ingredient preparation can be converted by adding small amounts of pharmaceutical excipients into a pharmaceutical preparation which can be converted directly, for example by compression, into a solid dosage form with a high ibuprofen concentration, optimal hardness and optimal active ingredient release.

Accordingly, the present invention further relates to the use of the novel active ingredient preparation for producing pharmaceutical preparations or solid dosage forms.

The novel pharmaceutical preparation comprises the novel ibuprofen-containing active ingredient preparation and, preferably, in addition tablet excipients which are adapted to the particular requirements for fast or slow active ingredient release or other pharmaceutical requirements.

The pharmaceutical preparation may, in addition to the active ingredient preparation, comprise further

binders apart from uncrosslinked PVP, such as, for example, hydroxypropylmethylcelluloses or hydroxypropylcelluloses,

disintegrants such as, for example, Ac-Di-Sol® supplied by FMC Corp., Philadelphia, Pa. 19103 or Primojel® supplied by VZB, Koog aan de Zaan, Foxkol, the Netherlands,

dry binders such as, for example, microcrystalline cellulose,

hydrophilicizing agents such as, for example, Cremophore RH 40 supplied by BASF Aktiengesellschaft, Ludwigshafen, Germany,

adsorbents such as, for example, Aerosile supplied by Degussa, Frankfurt, Germany,

lubricants such as, for example, magnesium stearate

or aids which bring about delayed release of the active ingredient, such as, for example, Kollidon^{(R)} SR supplied by BASF Aktiengesellschaft, Ludwigshafen, Germany or highly polymeric hydroxypropylmethylcellulose of the K4M or E4M type supplied by Colorcon, 65510 Idstein, Germany.

In a preferred embodiment, the pharmaceutical preparations comprise excipients which bring about delayed release of active ingredient from solid dosage forms.

The amount of excipients present in the pharmaceutical preparations in addition to the active ingredient preparation is not critical and, depending on the requirements, is typically for

| | |
|---|---|
| disintegrants | 0 to 5% by weight, |
| dry binders | 0 to 10 or 15% by weight, |
| hydrophilicizing agents | 0 to 1% by weight, |
| adsorbents | 0 to 1% by weight, |
| lubricants | 0 to 1% by weight, |

aids which bring about delayed release of the active ingredient 0 to 40% by weight,

while the amount of the novel active ingredient preparation in the pharmaceutical preparation is typically from 30 to 96% by weight, in each case based on the dry weight of the pharmaceutical formulation and thus also based on the dry weight of the solid dosage form produced therefrom.

The percentages by weight of the ingredients in the pharmaceutical preparation or the solid dosage forms add up to 100% by weight.

However, it is particularly advantageous to admix only small amounts of excipients to the active ingredient preparation in the pharmaceutical preparation, because this makes it possible to produce solid dosage forms with high active ingredient concentrations.

Depending on the ibuprofen concentration in the active ingredient preparation and the amount of added excipients in the pharmaceutical preparations it is possible to produce pharmaceutical preparations, and from them solid dosage forms, preferably tablets, with optimal hardness and optimal active ingredient release and having an ibuprofen concentration of greater than 80% by weight, in particular greater than 90% by weight or even greater than 94% by weight, in each case based on the dry weight of the pharmaceutical formulation or of the solid dosage form.

In these advantageous solid dosage forms the total of the proportions of binder or additives in the active ingredient preparation and of the excipients additionally admixed to the pharmaceutical preparation accordingly does not exceed 20% by weight and is, in particular, less than 10% by weight or even less than 6% by weight.

In addition to the ibuprofen-containing active ingredient preparation, it is possible for the pharmaceutical preparation and the solid dosage form produced therefrom to comprise, besides the excipients which are added where appropriate, further active ingredients such as, for example, pseudoephedrine hydrochloride or sulfate, codeine compounds such as dihydrocodeine hydrogen tartrate, or other active ingredients for which combination with ibuprofen is pharmacologically worthwhile.

The amount of added further active ingredient is not critical and may be, depending on the active ingredient and indication, typically from 1 to 80% by weight, based on the dry weight of the pharmaceutical formulation or of the solid dosage form.

A solid dosage form comprising a novel ibuprofen-containing active ingredient preparation and a codeine compound typically contains a codeine dose of 10 mg.

The novel pharmaceutical preparations comprising the novel ibuprofen-containing active ingredient preparation which are described above can be used directly for producing solid dosage forms.

Conversion of the pharmaceutical preparations into solid dosage forms can take place in a manner known per se by all conventional methods.

In a preferred embodiment of a process for producing solid dosage forms, the novel active ingredient preparation or the novel pharmaceutical preparation comprising the active ingredient preparation is compressed to a solid form. The novel pharmaceutical preparation comprising the novel active ingredient preparation is preferably compressed in a tablet mold, that is to say directly tableted.

In the direct tableting, the solid dosage forms produced from the novel pharmaceutical preparations have the same composition as the pharmaceutical preparation.

The resulting solid dosage forms may, where appropriate, be coated with coating agents such as, for example, films of Pharmacoat® 603 supplied by Shinetsu with polyethylene glycols as plasticizers.

Accordingly, the present invention further relates to solid dosage forms, preferably tablets, comprising the novel active ingredient preparation or the novel pharmaceutical preparation.

The novel solid dosage forms may have an ibuprofen concentration of more than 75%, in particular more than 90% by weight or even more than 94% by weight.

Compared with the prior art, the present invention has the following advantages:

The high ibuprofen concentration in the active ingredient preparations which can be tableted directly means that high ibuprofen concentrations can be achieved in the solid dosage forms.

The underlying active ingredient preparation is free from uncrosslinked PVP, so that superfast tableting is possible, and the resulting tablets are stable on storage.

The tablets compressed from the novel pharmaceutical preparations display, with only low compressive forces, a sufficiently high degree of hardness with negligible friability and a very rapid active ingredient release if no aids intended to delay release are present.

The novel active ingredient preparation can be tableted directly in combination with other active ingredients.

The novel pharmaceutical preparation can be used directly for producing tablets varying in size and thus be used flexibly for producing tablets with different ibuprofen doses.

The following examples illustrate the invention, the selection of the examples being non-limiting.

### EXAMPLE 1

Ibuprofen-Containing Active Ingredient Preparations

1.1 Hydroxypropylmethylcellulose (HPMC) as Binder

1.1.1 Coating by Wet Mixing and Drying

9.72 kg of ibuprofen (fine powder) were vigorously mixed with 240 g of hydroxypropylmethylcellulose and 40 g of Aerosil® 200 in a Lödige® mixer (granulator supplied by Lödige, Paderborn) and, with gentle stirring by the bottom propeller and the chopper at setting 1, moistened with 1.50 kg of distilled water and vigorously stirred for a further 5 minutes. The wet material was then forced through a sieve of mesh width 3 to 4 mm and dried in 2 portions in a fluidized bed drier (supplied by Glatt) at an inlet air temperature of 50 to 60° C. Sieving with sieves smaller than 800 μm was carried out. According to a laser diffraction particle measurement, agglomerates of less than 80 to 90 μm 5 were present only in proportions of a few percent, from 2 to 5%.

Composition of the active ingredient preparation 1.1.1, based on dry weight:

| | |
|---|---|
| Ibuprofen: | 97.2% by weight |
| Hydroxypropylmethylcellulose: | 2.4% by weight |
| Aerosil 200: | 0.4% by weight |

1.1.2 Coating by Fluidized Bed Granulation (Wurster Type)

2.375 kg of ibuprofen (fine powder) were vigorously mixed with 15 g of Aerosile 200 in a Stephan® mixer (granulator). Moistening with water resulted in a mixture which was slightly moist to the touch. This was firstly dried in a Glatt® fluidized bed granulator with a Wurster tube under inlet air at 50° C. and then sprayed with a solution of 110 g of HPMC of the Pharmacoat® 603 type, supplied by Shinetsu, in 2 kg of distilled water by bottom spraying of an amount of 75 g of solution per minute with inlet air at 50° C. Completion of the spraying was followed by drying until the product temperature was 38° C., and the material was forced through a sieve (Frewitt®) with a mesh width of 0.8 mm.

Composition of the active ingredient preparation 1.1.2, based on dry weight:

| | |
|---|---|
| Ibuprofen: | 95.0% by weight |
| Hydroxypropylmethylcellulose: | 4.4% by weight |
| Aerosil 200: | 0.6% by weight |

An active ingredient preparation 1.1.2.A consisting of 98.5% by weight of ibuprofen and 1.5% by weight of HPMC without Aerosil 200 was produced analogously.

An active ingredient preparation 1.1.2.B consisting of 97.5% by weight of ibuprofen and 2.5% by weight of HPMC without Aerosil 200 was produced analogously.

1.2 Hydroxypropylcellulose (HPC) as Binder

962 g of ibuprofen (fine powder) were vigorously mixed with 30 g of hydroxypropylcellulose of the Klucele type supplied by Hercules Corp., Milldate, Conn., USA and 8 g of Aerosil® 200 in a Stephan® laboratory mixer (granulator) and, with gentle stirring by stirrer device 1, moistened with 130 g of distilled water and vigorously mixed for a further 5 minutes. The moist material was then forced through a sieve of mesh width 3 to 4 mm and dried in a fluidized bed drier (supplied by Glatt) at an inlet air temperature of 50 to 60° C. A sieving with sieves smaller than 800 μm was carried out. According to a laser diffraction particle measurement, agglomerates below 80 to 90 μm were present in proportions of only a few percent, of 3 to 6%.

Composition of the active ingredient preparation 1.2, based on dry weight:

| | |
|---|---|
| Ibuprofen: | 96.2% by weight |
| Hydroxypropylcellulose: | 3.0% by weight |
| Aerosil 200: | 0.8% by weight |

An active ingredient preparation 1.2.A consisting of 98% by weight of ibuprofen and 2% by weight of HPC without Aerosil 200 was produced analogously.

1.3 Gelatin as Binder

4.5 kg of ibuprofen (fine powder) were vigorously mixed with 25 g of Aerosil® 200 in a Diosna® mixer (granulator). With gentle stirring by the bottom propeller and the chopper at setting 1, 500 g of distilled water were used for moistening, and the mixture was vigorously stirred for a further 5 minutes until ibuprofen and Aerosil could no longer be separated. The moist material was then placed in a fluidized bed granulator of the type supplied by Glatt and sprayed with an aqueous solution of 225 g of gelatin in 3.525 kg of water at 50° C., spraying amounts of 150 g/minute at an inlet air temperature of 60° C. Completion of spraying was followed by drying until the product temperature was 38° C., and the material was forced through a sieve (Frewitt®) with a mesh width of 0.8 mm.

Composition of the active ingredient preparation 1.3, based on dry weight:

| | |
|---|---|
| Ibuprofen: | 94.74% by weight |
| Gelatin: | 4.74% by weight |
| Aerosil 200: | 0.52% by weight |

An active ingredient preparation 1.3.A consisting of 99% by weight of ibuprofen and 1% by weight of gelatin without Aerosil 200 was produced analogously.

1.4 Sodium Carboxymethylcellulose (NaCMC) as Binder

9.78 kg of ibuprofen (fine powder, grade 25 supplied by Knoll AG, Ludwigshafen) were vigorously mixed with together with 20 g of Aerosil 200 and 200 g of Tylose® C (Na carboxymethylcellulose supplied by Hoechst, Frankfurt) in a Stephan laboratory mixer, and water was added in small amounts until the resulting composition was soil-moist (about 1.5 to 2.0 1 of water). The composition was reduced in size through a sieve with a mesh width of 3.15 mm (Frewitt®) and was dried in a Glatt® fluidized bed drier with inlet air at 50° C. The dry product was then again forced through a sieve with a mesh width of 0.8 mm (Frewitt^{(R)}).

Composition of the active ingredient preparation 1.4, based on dry weight:

| | |
|---|---|
| Ibuprofen: | 97.8% by weight |
| NaCMC: | 2.0% by weight |
| Aerosil 200: | 0.2% by weight |

An active ingredient preparation 1.4.A consisting of 98.5% by weight of ibuprofen and 1.5% by weight of NaCMC without Aerosil 200 was produced analogously.

1.5 Methylcellulose as Binder

4.275 kg of ibuprofen 38 (Knoll AG) were agglomerated with a solution of 0.203 kg of methylcellulose in 5 kg of distilled water in a fluidized bed at an inlet air temperature of 50° C. at a spraying rate of about 90 g/minute for about one hour, with a vibration period of about 30 seconds every 3 minutes. After drying to reach an outlet air temperature of about 40° C., 22 g of Aerosil 200 were admixed, and the product was forced through a sieve with a mesh width of 0.8 mm.

Composition of the active ingredient preparation 1.5, based on dry weight:

| | |
|---|---|
| Ibuprofen: | 95% by weight, |
| Methylcellulose: | 4.51% by weight, |
| Aerosil 200: | 0.49% by weight, |

An active ingredient preparation 1.5.A consisting of 98% by weight of ibuprofen and 2% by weight of methylcellulose without Aerosil 200 was produced analogously.

An active ingredient preparation 1.5.B consisting of 96% by weight of ibuprofen and 4% by weight of methylcellulose without Aerosil 200 was produced analogously.

### EXAMPLE 2

Production of Compacts from the Active Ingredient Preparation and Comparison of the Data with Compacts Produced from Physical Mixtures of the Same Composition.

Various compositions of the novel active ingredient preparation were compressed under various pressures using a type EKO press supplied by Korsch, Berlin, to give 200 mg compacts with a diameter of 9 mm. For comparison therewith, the physical mixtures (PM) of the same composition were compressed under analogous conditions, and the hardnesses of the produced compacts were compared. Physical mixtures mean the dry mixture of ibuprofen with the particular binder.

The results are listed in the following table. In contrast to the purely physical mixtures, the novel ibuprofen-containing active ingredient compositions result, even with low compressive forces, in compacts with hardnesses which are distinctly above the hardnesses of 25 N which are adequate for tablets.

TABLE
Dependence of the hardness of ibuprofen compacts on the
particular pressure and on comparison of novel active
ingredient preparation (AP) and physical mixture (PM)

| | | Hardness in [N] | | |
|---|---|---|---|---|
| | Preparation of | with a compressive force | | |
| Composition | example | of | | |
| Ibuprofen with | | 4.7 to | 9.7 to | 20.1 to |
| quantity of binder | | 5.3 kN | 10.9 kN | 21.1 kN |
| in [% by wt] based | | | | |
| on composition | | | | |
| 0% (pure) | — | 11.0 | 10.8 | 20.1 |
| 1.5% HPMC | AP 1.1.2.A | 63.0 | 120.0 | 165.0 |
| | PM | 13.0 | 18.0 | 30.0 |
| 2.5% HPMC | AP 1.1.2.B | 70.0 | 131.0 | 160.0 |
| | PM | 15.0 | 19.0 | 29.0 |
| 2% methyl- | AP 1.5.A | 85.0 | 145.0 | 153.0 |
| cellulose | PM | 9.0 | 16.0 | 38.0 |
| 4% methyl- | AP 1.5.B | 98.0 | 148.0 | 175.0 |
| cellulose | PM | 14.0 | 20.0 | 29.0 |
| 2% HPC | AP 1.2.A | 65.0 | 98.0 | 156.0 |
| | PM | 18.0 | 21.0 | 35.0 |
| 1.5% NaCMC | AP 1.4.A | 98.0 | 130.0 | 180.0 |
| | PM | 8.0 | 15.0 | 28.0 |
| 1% gelatin | AP 1.3.A | 53.0 | 98.0 | 121.0 |
| | PM | 7.0 | 12.0 | 30.0 |

Abbreviations:
HPMC = hydroxypropylmethylcellulose
HPC = hydroxypropylcellulose
NaCMC = sodium carboxymethylcellulose

### EXAMPLE 3

Production of Tablets from the Active Ingredient Preparations

The active ingredient preparations produced in Example 1 were mixed with further excipients and/or further active ingredients (batch size about 1 kg) and compressed directly to tablets in an instrumented EK 0 type eccentric press supplied by Korsch, Berlin. The following tables in the examples indicate the composition of the pharmaceutical preparation for a tablet and thus the composition of the specific tablet.

3.1 Ibuprofen Tablet with a Dose of 200 mg of Active Ingredient from the Active Ingredient Preparation of Example 1.2

| Active ingredient | 208 mg |
|---|---|
| preparation of Example | (corresponds to 200 mg of |
| 1.2 | ibuprofen) |
| Lactose | 10.0 mg |
| Pregelatinized starch | 15.0 mg |
| AcDiSol | 12.0 mg |
| Magnesium stearate | 1.5 mg |
| Aerosil | 0.5 mg |
| Tablet weight: | 247 mg |
| Punch size: | 9 mm round |
| Active ingredient content: | 81% by weight |
| Compressive force: | 6 kN |
| Hardness: | 89 N |
| Disintegration: | 2 minutes, beaker with |
| | water at room temperature |
| Active ingredient release: | 5 minutes 78% |
| | 10 minutes 98% (USP method) |

3.2 Ibuprofen Tablet with a Dose of 600 mg of Active Ingredient from the Active Ingredient Preparation of Example 1.2

Compression of tablets with the same formula as in Example 3.1 in an oblong format of 6.5×18 mm and with a weight of 741 mg under a compressive force of 7 kN results in tablets with a hardness of 158 N and a disintegration time of 2 minutes in water. The active ingredient release by the USP XXII method shows 100% ibuprofen dissolved after 10 minutes.

3.3 Ibuprofen Tablet with a Dose of 600 mg of Active Ingredient from the Active Ingredient Preparation of Example 1.1.1

| Active ingredient | 617 mg |
|---|---|
| preparation of Example | (corresponds to 600 mg of |
| 1.1.1 | ibuprofen) |
| Microcrystalline | 18.0 mg |
| cellulose | |
| Lactose | 6.0 mg |
| AcDiSol | 14.0 mg |
| Magnesium stearate | 4.5 mg |
| Aerosil | 1.5 mg |
| Tablet weight: | 661 mg |
| Punch size: | 6.5 × 18 mm oblong |
| Active ingredient content: | 91.1% by weight |
| Compressive force: | 9 kN |
| Hardness: | 180 N |
| Disintegration: | 4 minutes, beaker with |
| | water at roon temperature |
| Active ingredient release: | 5 minutes 68% |
| | 10 minutes 89% |
| | 15 minutes 99% (USP method) |

3.4 Ibuprofen Tablet with a Dose of 400 mg of Active Ingredient from the Active Ingredient Preparation of Example 1.1.1

Compression of tablets with the same formula as in Example 3.3 with a punch format of 11 mm round and with a weight of 440.8 mg under a compressive force of 6 kN results in tablets with a hardness of 138 N and a disintegration time of 3 minutes in water. The active ingredient released by the USP XXII method shows 100% ibuprofen dissolved after 15 minutes.

3.5 Combination Tablet with a Dose of 400 mg of Ibuprofen and 10 mg of Dihydrocodeine Hydrogen Tartrate from the Ibuprofen-Containing Active Ingredient Preparation of Example 1.1.1

| Active ingredient | 411.5 mg |
|---|---|
| preparation of Example | (corresponds to 400 mg of |
| 1.1.1 | ibuprofen) |
| Dihydrocodeine | 10 mg |
| hydrogen tartrate | |
| Microcrystalline | 25 mg |
| cellulose | |
| Lactose | 10 mg |
| Pharmacoat ® 603 | 8 mg |
| AcDiSol | 12 mg |
| Magnesium stearate | 3 mg |
| Aerosil | 1 mg |
| Tablet weight: | 480.5 mg |
| Punch size: | 11 mm round |
| Active ingredient content: | 86.3% by weight |
| Compressive force: | 6 kN |
| Hardness: | 95 N |
| Disintegration: | 3 minutes, beaker with |
| | water at room temperature |
| Release of active | 5 minutes 68% |
| ingredient ibuprofen: | 10 minutes 89% |
| | 15 minutes 99% (USP method) |

3.6 Ibuprofen Tablet with a Dose of 200, 400, 600 and 800 mg of Active Ingredient from the Active Ingredient Preparation of Example 1.1.1 with an Active Ingredient Content of 80% by Weight in the Tablet

| Active ingredient | 83.3% by weight |
|---|---|
| preparation of Example | (corresponds to 80% by weight of |
| 1.1.1 | ibuprofen) |
| Microcrystalline | 4.3% by weight |
| cellulose | |
| Lactose | 5.4% by weight |
| AcDiSol | 6.0% by weight |
| Aerosil 200 | 0.6% by weight |
| Magnesium stearate | 0.4% by weight |

The pharmaceutical preparations mixed from these ingredients were compressed to tablets with a weight of 250 mg (=200 mg of ibuprofen), 500 mg (=400 mg of ibuprofen), 750 mg (=600 mg of ibuprofen) and 1000 mg (=800 mg of ibuprofen) in tablet sizes of 9 mm round, 11 mm round, 6.5×18 mm and 7.5×21 mm. All the tablets release the active ingredient ibuprofen quantitatively in 10 minutes.

We claim:

1. A process for producing an active ingredient preparation comprising ibuprofen, wherein ibuprofen or its salt is coated with hydroxypropylmethylcellulose, methylcellulose, sodium carboxymethylcellulose, hydroxypropylcellulose or gelatin as binder, where the proportion of the binder is from 0.1 to 10% by weight and the proportion of ibuprofen or its salts is from 90 to 99.9% by weight, based on the dry weight of the active ingredient preparation.

2. A process as claimed in claim 1, wherein from 0.1 to 1.5% by weight of highly disperse silica are added to the ibuprofen or its salts to be coated, based on the dry weight of the active ingredient preparation.

3. An active ingredient preparation obtainable by a process as claimed in either of claims 1 or 2.

4. An active ingredient preparation consisting of

| | |
|---|---|
| 90.0 to 99.9% | by weight of ibuprofen or its salts, |
| 0.1 to 10.0% | by weight of hydroxypropyl- |
| | methylcellulose, methylcellulose, |
| | sodium carboxymethylcellulose, |
| | hydroxypropylcellulose or gelatin as |
| | binder |
| | and |
| 0.1 to 1.5% | by weight of highly disperse silica, |

in each case based on the dry weight of the active ingredient preparation, where
the compacts produced from this active ingredient preparation with a compressive force of from 4.7 to 5.3 kN and having an ibuprofen active ingredient content of 200 mg and a diameter of 9 mm have a hardness of at least 25 N.

5. The use of the active ingredient preparation as claimed in claim 3 or 4 for producing pharmaceutical preparations or solid dosage forms.

6. A pharmaceutical preparation comprising an active ingredient preparation as claimed in claim 3 or 4.

7. A pharmaceutical preparation as claimed in claim 6, wherein the pharmaceutical preparation comprises tablet excipients in addition to the active ingredient preparation.

8. A pharmaceutical preparation as claimed in claim 7, wherein the substances present as tablet excipients bring about delayed release of active ingredient from solid dosage forms.

9. A pharmaceutical preparation as claimed in any of claims 6 to 8, which additionally comprises further active ingredients.

10. The use of the pharmaceutical preparations as claimed in any of claims 6 to 9 for producing solid dosage forms.

11. A process for producing solid dosage forms, which comprises compressing an active ingredient preparation as claimed in claim 3 or 4 or a pharmaceutical preparation as claimed in any of claims 6 to 9 to a solid form and, where appropriate, coating this solid dosage form with coating agents.

12. A solid dosage form comprising an active ingredient preparation as claimed in claim 3 or 4.

13. A solid dosage form comprising a pharmaceutical preparation as claimed in any of claims 6 to 9.

14. A solid dosage form as claimed in claim 12 or 13 with an ibuprofen content of more than 75% by weight.
